(19) United States
(12) Patent Application Publication
(10) Pub. No.: US 2007/0267014 A1
(43) Pub. Date: Nov. 22, 2007

(54) Sterilizing facemask with a multi-layer filter

(76) Inventor: Jing-Jyr Lin, Taoyuan County (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/474,276

(22) Filed: Jun. 26, 2006

Related U.S. Application Data
(63) Continuation-in-part of application No. 11/437,788, filed on May 22, 2006.

(51) Int. Cl. A61M 16/00

(52) U.S. Cl. 128/203.11

(57) ABSTRACT

A sterilizing facemask with a multi-layer filter discloses a first sterilizing layer and a second sterilizing layer. The first sterilizing layer provides inhaled aseptic air such as using ultraviolet light or visible light in external light hydroxyl free radicals to sterilize. The second sterilizing layer provides exhaled aseptic air from human such as using inactivated the enzyme of bacteria's sulfhydryl group to sterilize. In addition, in accordance with types of germs carried by users, the material of the second sterilizing layer can be further modified to ensure that the exhaled air is sterilized.

### FIELD OF THE INVENTION

The present invention relates generally to a facemask, and particularly to a sterilizing facemask that can sterilize inhaled and exhaled air bidirectionally.

### BACKGROUND OF THE INVENTION

Owing to development of industries, considerable quantities of exhaust gases by industries and traffic vehicles are generated. Accordingly, in order to let people breathe fresher air, some products emerge to meet the demand. Nowadays, the filtering layers of an air purifier and facemasks used by people have the function of filtering air. The structure and design of the facemask disclosed in U.S. Pat. No. 6,520,181 emphasizes in preventing gases from leaking out without special adhesion function. The general planar foldable facemask disclosed in U.S. Pat. No. 4,941,470 only contains a layer of meltblown polypropylene fabrics without the effects of suppressing bacteria and of molecule adhesion, and thereby cannot be used in environments with high air pollution and with high bacteria pollution. The three-layer mask with activated charcoal disclosed in U.S. Pat. No. 6,070,578 has the effect of molecule adhesion but has no bacteria suppressing effect. Taiwan patent number 163,571 discloses using photocatalyzers to resist bacteria. However, the photocatalyzers tend to depart or peel off from the non-woven fabrics or textiles and to be inhaled into lungs, causing danger to people. In addition, because the photocatalyzers is on the middle layer of the facemask, it cannot absorb ultraviolet light and thereby the bacteria suppressing effect is reduced. Furthermore, the planar facemask disclosed in Taiwan patent number 154,980 has no bacteria suppressing and molecule adhesion effects.

Ordinary facemasks are designed for filtering inhaled external air, and there are plenty of materials used, for example, photocatalyzers, nanometer silver, and chitin. Nevertheless, they are single-layered, and thereby cannot filter exhaled air to avoid germs from spreading by a carrier. Photocatalyzers can be used when air comes from outside. Nevertheless, when exhaling air, one side of the facemasks contacts with skins and thereby cannot be illuminated by visible light or ultraviolet light. Hence, photocatalyzing effect is bad or even ineffective to sterilize both exhaled and inhaled air. Accordingly, the present invention provides a novel structure of facemasks for providing distinct sterilizing mechanisms on exhaled and inhaled air.

### SUMMARY

The purpose of the present invention is to provide a sterilizing facemask with a multi-layer filter for providing distinct sterilizing mechanisms on exhaled and inhaled air for users, and thereby to provide a facemask with better sterilizing effects on exhaled and inhaled air.

The other purpose of the present invention is to provide a sterilizing facemask with a multi-layer filter for increasing the pressure drop inside the facemask and thereby further provide an increased respiration zone for the facemask structure.

In order to achieve the purposes and effects described above, the present invention provides a sterilizing facemask with a multi-layer filter, which discloses a first sterilizing layer and a second sterilizing layer. The first sterilizing layer uses ultraviolet light or visible light in external light for providing hydroxyl free radicals to sterilize inhaled air. Because the second sterilizing layer is inside of the facemask, there is no light provided. The second sterilizing layer inactivates the enzyme of bacteria's sulfhydryl group to sterilize exhaled air. In addition, in accordance with types of germs carried by users, the material of the second sterilizing layer can be further modified to ensure that the exhaled air is sterilized.

### BRIEF DESCRIPTION OF THE DRAWINGS

FIG. 1 is a structural schematic diagram according to a preferred embodiment of the present invention;

FIG. 2 is a front view of a facemask with a shelter layer according to another preferred embodiment of the present invention;

FIG. 3 is a structural schematic diagram of a facemask with a function layer according to another preferred embodiment of the present invention;

FIG. 4 is a structural schematic diagram of a three-dimensional facemask with sterilizing functions according to another preferred embodiment of the present invention; and

FIG. 5 is a structural schematic diagram of a facemask with sterilizing functions according to another preferred embodiment of the present invention.

### DETAILED DESCRIPTION

In order to make the structure and characteristics as well as the effectiveness of the present invention to be further understood and recognized, the detailed description of the present invention is provided as follows along with preferred embodiments and accompanying figures.

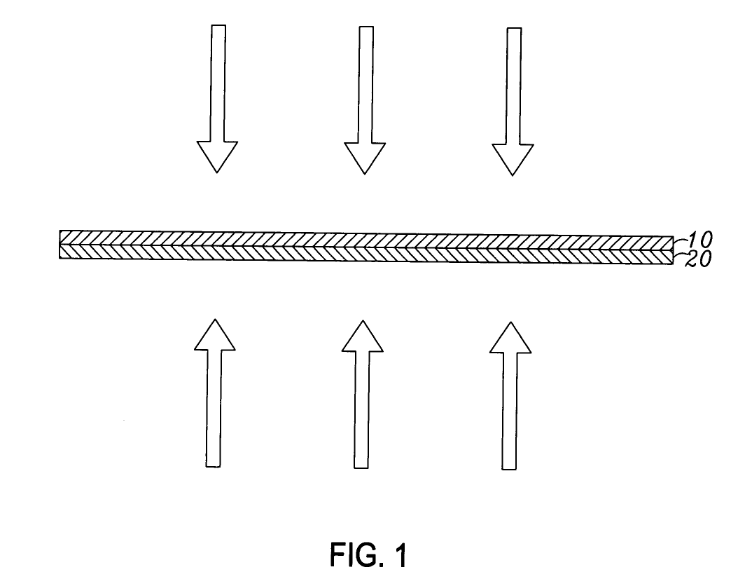

FIG. 1 is a structural schematic diagram according to a preferred embodiment of the present invention. As shown in the figure, the present invention discloses a multi-layer sterilizing facemask, which includes a first sterilizing layer 10, a second sterilizing layer 20. The first sterilizing layer 10 is adapted on one side of the second sterilizing layer 20 for providing better filtering effects by sterilizing inhaled and exhaled air in two distinct mechanisms. In accordance with types of germs carried by users, the material of the second sterilizing layer 20 can be further modified to sterilize the exhaled air.

Because surface electrons on the first sterilizing layer 10 can absorb sufficient energy to escape when the first sterilizing layer 10 is illuminated by external light, no matter ultraviolet light or visible light, holes will be formed on the locations from which the electrons escape. The holes will oxidize (that is, capture the electrons thereof hydroxyl group (OH—) that is ionized from neighboring water molecules and make the OH— become very active hydroxyl free radicals. When the hydroxyl free radicals meet organic materials, they will capture the electrons back, and the organic molecules will break, disintegrate, and decompose. Most of common pollutions or pathogens are carbohydrates, which will mostly become non-harmful water and carbon dioxide, and thereby the purposes of decontamination and sterilization can be achieved. The material of the first sterilizing layer 10 is chosen from the group consisting of TiO₂, ZnO, SnO₂, ZrO₂, CdS, ZnS, and sulfides. Furthermore, because the second sterilizing layer 20 cannot be illuminated by visible light or ultraviolet light, the material thereof cannot be chosen as the same material as the first sterilizing layer 10. The material of the second sterilizing layer 20 is chosen from chitin or silver ions. In addition, according to types of germs carried by users, the material of the second sterilizing layer 20 is different.

Chitin is a copious natural resource. It is manufactured using biosynthesis by one billion tons each year, and is the most abundant natural organic matter second to cellulose on earth. The structure of chitin is very similar to cellulose. It is a natural polysaccharide, and is named as (1,4)-2-acetamino-2-deoxy-β-D-glucose. Chitin has the biological functions of collagen in tissues of higher-grade animals, and of cellulose in tissues of higher-grade plants, thereby it has fine adaptation for both animals and plants. In addition, it is biologically degradable and oral non-poisonous. Thereby, nowadays, it has become a new material with wide applications. Chitosan is the product of chitin after deacetylation treatment, and is solvable in solutions with low acidity. Because it contains ionized amine group to combine with acid molecules, it has many special physical and chemical properties, as well as exceptional biological functions. Chitosan is the most important derivative of chitin and is the product of chitin after deacetylation above 70%. It is, so far, the only natural alkaline polysaccharide with the properties of non-toxicity, biological degradability, and good biological compatibility. Chitin can eliminate over 99% of pathogenic toxicity, and will not produce antibodies and cause allergic reactions. In addition, it also has the properties of moisture holding and non-toxicity.

The sterilizing mechanism of silver is when nanometer silver approaches virus, fungus, bacteria, or bacteriophage, it will cause their protein enzyme, which is responsible for oxygen metabolism, decompose and lose its effect. Hence, the virus, fungus, bacteria, or bacteriophage cannot carry out normal oxygen metabolism and thereby will die naturally.

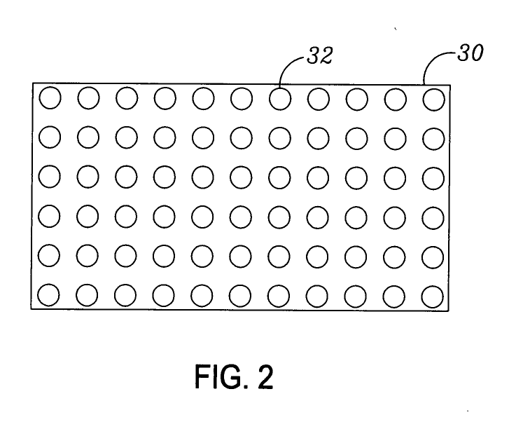

FIG. 2 is a front view of a facemask with a shelter layer according to another preferred embodiment of the present invention. As shown in the figure, the present invention further installs a shelter layer 30 between the first sterilizing layer and the second sterilizing layer. The material thereof can be chosen from damp-proof materials or water-repellent materials. A plurality of holes 32 is adapted outside of the shelter layer 30 for providing visible light or ultraviolet light.

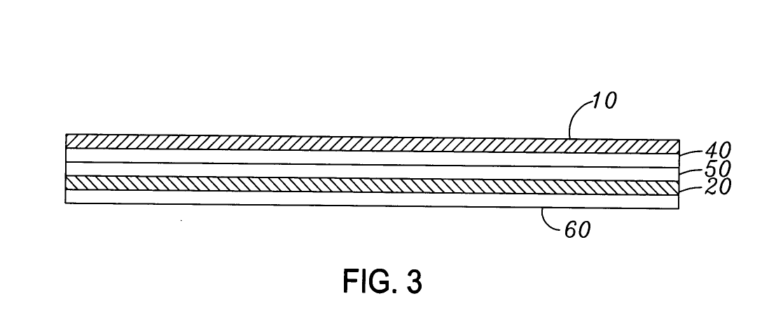

FIG. 3 is a structural schematic diagram of a facemask with a function layer according to another preferred embodiment of the present invention. As shown in the figure, the present invention further includes an odor-removal layer 40, an electrostatic layer 50, and a skin contact layer 60. The odor-removal layer 40 and the electrostatic layer 50 are adapted between the first sterilizing layer 10 and the second sterilizing layer 20; the order of the odor-removal layer 40 and the electrostatic layer 50 can be swapped. In addition, the odor-removal layer 40 can be made of activated charcoal; the skin contact layer 60 is adapted inside of the second sterilizing layer 20.

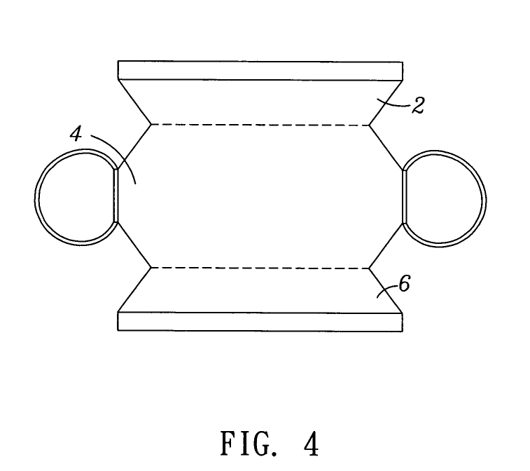

FIG. 4 is a structural schematic diagram of a three-dimensional facemask with sterilizing functions according to another preferred embodiment of the present invention. As shown in the figure, the facemask constructed by the first sterilizing layer and the second sterilizing layer according to the present invention includes an upper surface 2, which is located at the upper part of the facemask, a center surface 4, which is located at the center of the facemask and below the upper surface, and a lower surface 6, which is located at the lower part of the facemask and is below the center surface 4. The upper, center, and the lower surfaces form an integral unit of surface. First, fold inwardly the upper surface 2 and the lower surface 6 besides the center surface 4, respectively. When using the facemask, the upper surface 2 and the lower surface 6 can extend to support the center surface 4 and to form a sterilizing facemask.

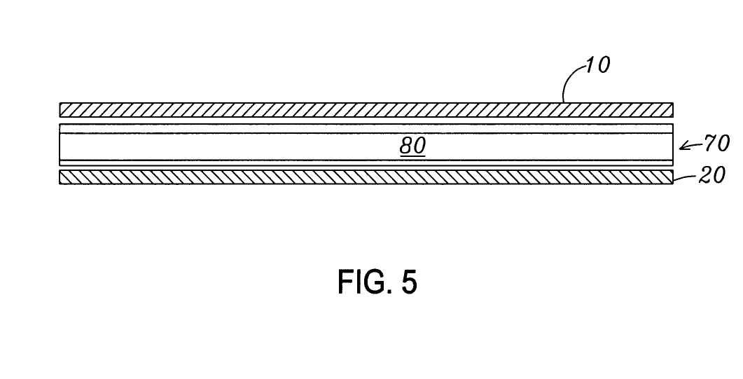

FIG. 5 is a structural schematic diagram according to another preferred embodiment of the present invention. As shown in the FIG. 5, the present invention discloses a multi-layer sterilizing facemask, which comprises a first sterilizing layer 10 and a second sterilizing layer 20, which adapted inside of the first sterilizing layer 10. The first sterilizing layer 10 and the second sterilizing layer 20 is applied for sterilizing inhaled external air and exhaled internal air from human bodies respectively. Likewise, the present invention further includes a decelerating layer 70 adapted between the first sterilizing layer 10 and the second sterilizing layer 20. The decelerating layer 70 is utilized for extended the sterilizing effect to improve the sterilizing effect, whereby it should be made the inhaled external air and the exhaled internal air from human bodies both slow down to pass through the multi-layer sterilizing facemask. Moreover, the deceleration layer 70 further includes a electrostatic layer 80, which make the sterilizing effect better, even more. Hence, the present invention should let people live better, whereby it is sterilizing the inhaled external air and the exhaled internal air from human bodies.

Accordingly, the present invention conforms to the legal requirements owing to its novelty, unobviousness, and utility. However, the foregoing description is only a preferred embodiment of the present invention, not used to limit the scope and range of the present invention. Those equivalent changes or modifications made according to the shape, structure, feature, or spirit described in the claims of the present invention are included in the appended claims of the present invention.

1. A sterilizing facemask with a multi-layer filter comprising:
a first sterilizing layer provided hydroxyl free radicals via external light; and
a second sterilizing layer adapted inside of the first sterilizing layer and inactivating the enzyme of bacteria's sulfhydryl group;
wherein inhaled external air is sterilized by the first sterilizing layer; and exhaled air from human bodies is sterilized by the second sterilizing layer.

2. The sterilizing facemask of claim 1, further comprising a shelter layer adapts outside of the first sterilizing layer.

3. The sterilizing facemask of claim 2, wherein the shelter layer includes damp-proof materials.

4. The sterilizing facemask of claim 2, wherein the shelter layer includes water-repellent materials.

5. The sterilizing facemask of claim 2, wherein the shelter layer includes a plurality of holes for providing light to the first sterilizing layer.

6. The sterilizing facemask of claim 1, further comprising an odor-removal layer adapts between the first sterilizing layer and the second sterilizing layer.

7. The sterilizing facemask of claim 6, wherein the odor-removal layer includes activated charcoal.

8. The sterilizing facemask of claim 1, further comprising an electrostatic layer adapts between the first sterilizing layer and the second sterilizing layer.

9. The sterilizing facemask of claim 1, further comprising a skin contact layer adapts inside of the second sterilizing layer.

10. The sterilizing facemask of claim 1, wherein the material of the first sterilizing layer is selected from the group consisting of TiO₂, ZnO, SnO₂, ZrO₂, CdS, ZnS, and sulfides.

11. The sterilizing facemask of claim 1, wherein the material of the second sterilizing layer is selected from silver ions or chitin.

12. The sterilizing facemask of claim 1, and further comprising:
an upper surface, located at the upper part of the facemask;
a center surface, located at the center of the facemask and below the upper surface; and
a lower surface, located at the lower part of the facemask and below the center surface;
the upper, center, and the lower surfaces forming an integral unit of surface; first, folding inwardly the upper surface and the lower surface besides the center surface, respectively; and when using the facemask, the upper surface and the lower surface extending to support the center surface and to form a sterilizing facemask.

13. A sterilizing facemask with a multi-layer filter comprising:
a first sterilizing layer; and
a second sterilizing layer adapted inside of the first sterilizing layer;
wherein inhaled external air is sterilized by the first sterilizing layer; and exhaled air from human bodies is sterilized by the second sterilizing layer.

14. The sterilizing facemask of claim 13, further comprising a deceleration layer adapts between the first sterilizing layer and the second sterilizing layer.

15. The sterilizing facemask of claim 14, wherein the deceleration layer includes a electrostatic layer.
